(19) United States
(12) Patent Application Publication
(10) Pub. No.: US 2009/0131465 A1
(43) Pub. Date: May 21, 2009

(54) Synthesis and crystalline forms of melanocortin-4 receptor agonist

(76) Inventors: Alex M. Chen, Edison, NJ (US); Jennifer R. Foley, Garwood, NJ (US); John Limanto, Rahway, NJ (US); Robert M. Wenslow, Cream Ridge, NJ (US); Karen C. Thompson, Lansdale, PA (US); Andrey V. Peresypkin, Cambridge, MA (US)

Correspondence Address: MERCK AND CO., INC, P O BOX 2000, RAHWAY, NJ, 07065-0907, US

(21) Appl. No.: 12/290,062

(22) Filed: Oct. 27, 2008

Related U.S. Application Data
(60) Provisional application No. 61/001,005, filed on Oct. 30, 2007.

(51) Int. Cl. A61K 31/438; C07D 401/06; A61P 3/04; A61P 3/10; A61P 15/10

(52) U.S. Cl. 514/278; 546/17

(57) ABSTRACT

The present invention relates to a process for producing crystalline N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide, and novel salts, solvates, hydrates and polymorphs thereof.

### BACKGROUND OF THE INVENTION

Compound I, N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide, is disclosed in U.S. Patent Publication No. US 2006/0183904 and PCT patent publication WO 2004/089307.

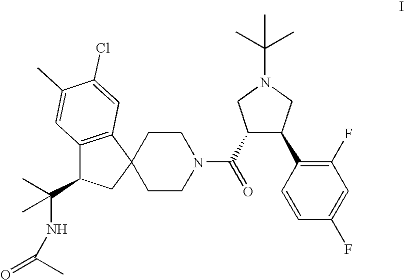

Compound I, and its novel polymorphic forms, are melanocortin-4 receptor agonists useful to treat melanocortin-4 receptor (MC4R) mediated diseases including, but not limited to, obesity, diabetes, male sexual dysfunction, erectile dysfunction, and female sexual dysfunction.

The final coupling step disclosed in US 2006/0183904 and WO 2004/089307 employed HATU, HOAT and 4-methyl morpholine in dichloromethane to couple an acid and an amine. The process of the present invention reacts an acid chloride and an amine in the final coupling step, resulting in cleaner and more economical amide formation with >99% conversion. The original process for the preparation of Compound I, as disclosed in US 2006/0183904 and WO 2004/089307, yielded Compound I as a trifluoroacetic acid (TFA) salt. There is no specific discussion or preparation of free base solvates or HCl salts of Compound I, or a hydrates or solvates thereof, in US 2006/0183904 and WO 2004/089307.

### SUMMARY OF THE INVENTION

This invention provides a process for producing crystalline N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I). This invention further provides four novel crystalline forms of N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (compound I) that have been identified, which are designated: 1) free base isopropyl alcohol solvate; 2) anhydrous mono HCl salt; 3) mono HCl salt hydrate; and 4) mono HCl salt acetonitrile solvate. The crystalline forms of these polymorphs are new and may have advantages in the preparation of pharmaceutical compositions of Compound I, such as ease of processing, handling and dosing. In particular, the crystalline anhydrous mono HCl salt of compound I has good aqueous solubility and good chemical and physical stability making it particularly suitable for the manufacture of pharmaceutical dosage forms.

The present invention also relates to pharmaceutical formulations comprising the novel polymorphs, solvates, hydrates and salts of compound I as active pharmaceutical ingredients, as well as methods for using them as melanocortin-4 receptor agonists in the treatment of melanocortin-4 receptor mediated disorders.

### BRIEF DESCRIPTION OF THE FIGURES

The invention is described with reference to the figures in which:

FIG. 1 is a characteristic X-ray diffraction pattern of the crystalline anhydrous mono HCl salt of Compound I;

FIG. 2 is a carbon-13 cross-polarization magic-angle spinning (CPMAS) nuclear magnetic resonance (NMR) spectrum of the crystalline anhydrous mono HCl salt of Compound I;

FIG. 3 is a typical DSC curve of the crystalline anhydrous mono HCl salt of Compound I;

FIG. 4 is a typical thermogravimetric (TG) curve of the crystalline anhydrous mono HCl salt of Compound I;

FIG. 5 is a characteristic X-ray diffraction pattern of the crystalline HCl salt hydrate of Compound I;

FIG. 6 is a characteristic X-ray diffraction pattern of the crystalline HCl salt acetonitrile solvate of Compound I; and

FIG. 7 is a characteristic X-ray diffraction pattern of the crystalline free base isopropyl alcohol solvate of Compound I.

### DETAILED DESCRIPTION OF THE INVENTION

The present invention provides a process for the preparation of crystalline N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide of structural formula I

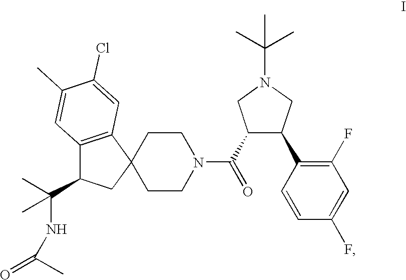

and polymorphs, solvates, hydrates, and salts thereof.

Crystal forms are convenient for the isolation of compound I with an upgrade in purity and represent a convenient scalable way to produce high purity Compound I. Four crystalline forms were identified including the crystalline free base of Compound I as an isopropyl alcohol solvate and three crystalline HCl salt forms of Compound I. These crystalline salts of compound I are novel and have improved physiochemical properties, such as purity, stability and ease of formulation that render them particularly suitable for the manufacture of pharmaceutical dosage forms. Discovery of crystalline forms allowed for the facile purification, isolation, and formulation of compound I.

The compound I can be prepared by employing the following General Scheme, which shows the formation of Compound I via the reaction of pyrrolidine II with spiropiperidine III.

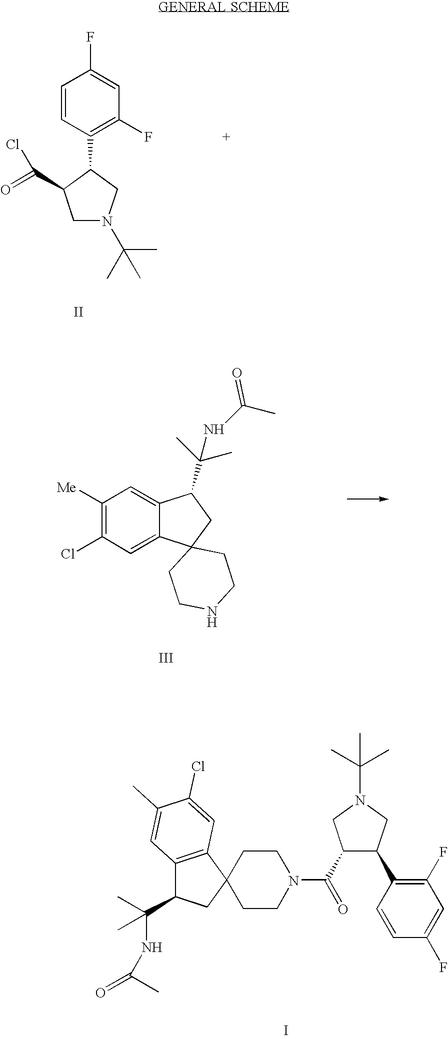

U.S. Patent Publication No. US 2006/0183904 and International patent publication WO 2004/089307 disclose the preparation of pyrrolidine acid IV and the preparation of Boc spiropiperidine V. Pyrrolidine IV may be easily converted to pyrrolidine II by converting the acid of pyrrolidine IV into an acid chloride.

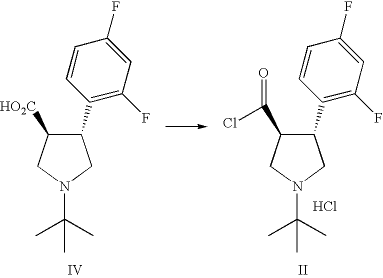

The Boc protected spiropiperidine V may be readily converted to free spiropiperidine III by removing the Boc protecting group.

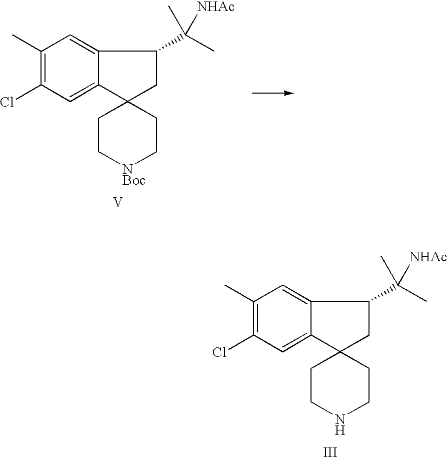

The HCl salt of compound I has four known crystalline polymorphs denoted as the freebase isopropyl alcohol solvate, the anhydrous mono HCl salt, the mono HCl salt hydrate and the mono HCl salt acetonitrile solvate.

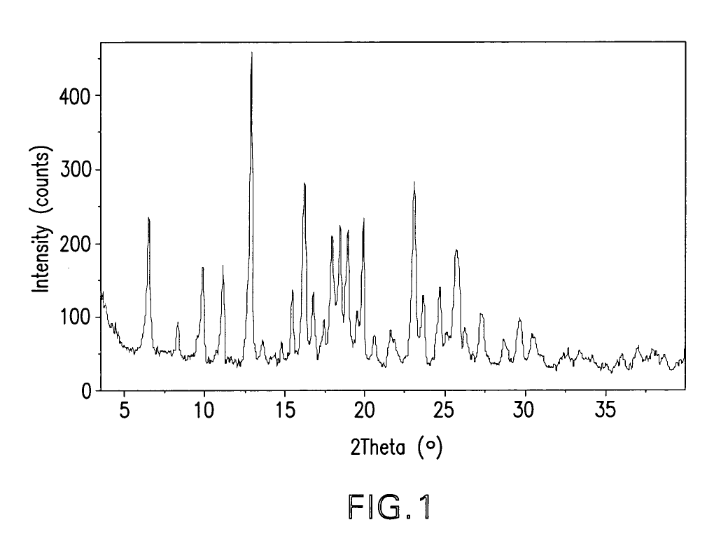

The X-ray diffraction pattern for the crystalline anhydrous HCl salt of compound I is shown in FIG. 1. The crystalline anhydrous HCl salt exhibited characteristic reflections corresponding to d-spacings of 13.58, 6.89, 5.46, 8.97, 7.96, 4.46, 3.86, 3.47, and 3.02 angstroms.

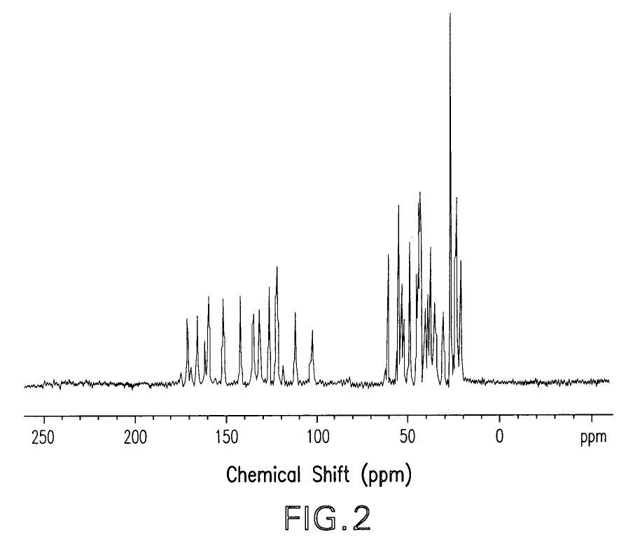

The solid-state carbon-13 CPMAS NMR spectrum for the crystalline anhydrous HCl salt of compound I is shown in FIG. 2. The crystalline anhydrous HCl salt exhibited characteristic signals with chemical shift values of 26.8, 43.1, 122.2, 159.6, 23.8, 43.8, 126.5, 171.1, 55.1, 151.7, and 131.6 p.p.m.

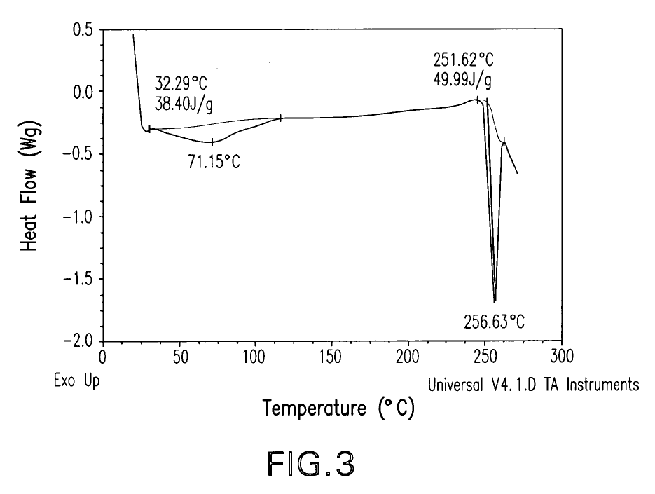

The differential calorimetry scan for the crystalline anhydrous HCl salt of compound I is shown in FIG. 3. The crystalline anhydrous HCl salt exhibited an endotherm due to melting and decomposition with an onset temperature of 251.6° C., a peak temperature of 256.6° C., and an enthalpy change of 50.0 J/g. It also exhibited another endotherm due to surface water coming out with an extrapolated onset temperature of 32.3° C., a peak temperature of 71.1° C., and an enthalpy change of 38.4 J/g.

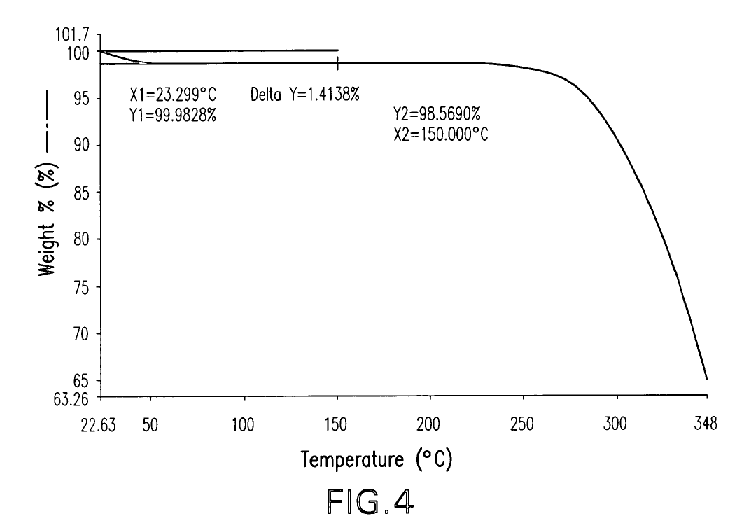

A characteristic thermogravimetric analysis (TGA) curve for the crystalline anhydrous HCl salt of compound I is shown in FIG. 4. TGA indicated a weight loss of about 1.4% due to surface water from ambient temperature to about 150° C.

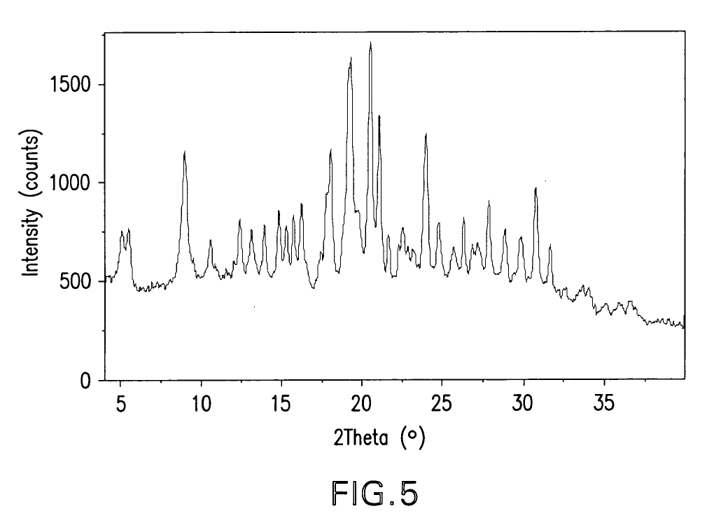

The X-ray diffraction pattern for the crystalline HCl salt hydrate of compound I is shown in FIG. 5. The crystalline HCl salt hydrate exhibited characteristic reflections corresponding to d-spacings of 9.86, 4.33, 3.71, 4.22, 3.20, 2.91, 7.16, 5.98, and 5.47 angstroms.

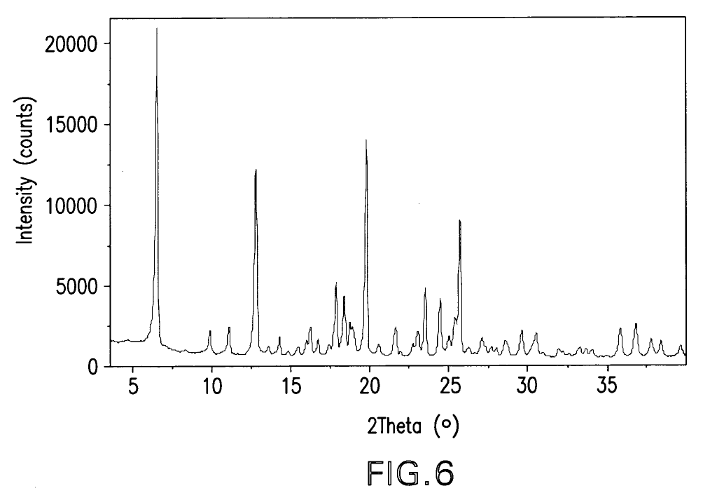

The X-ray diffraction pattern for the crystalline HCl salt acetonitrile solvate of compound I is shown in FIG. 6. The crystalline HCl salt acetonitrile solvate exhibited characteristic reflections corresponding to d-spacings of 13.58, 6.92, 5.46, 8.95, 7.99, 4.48, 3.77, 3.46, and 3.02 d-spacings of angstroms.

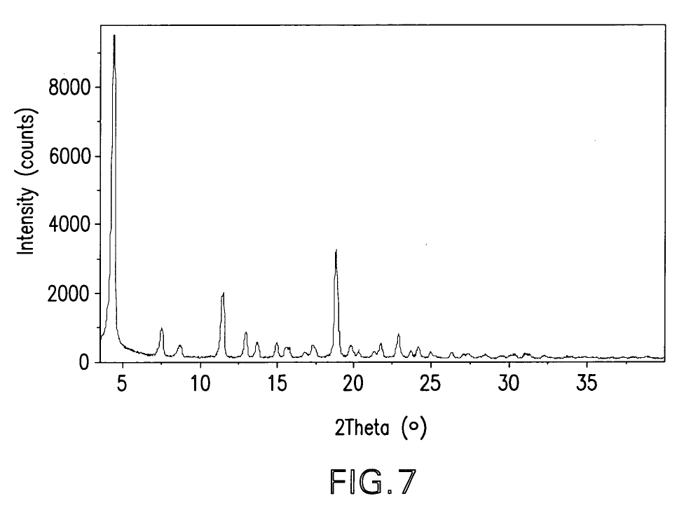

The X-ray diffraction pattern for the crystalline free base isopropyl alcohol solvate of Compound I is shown in FIG. 7. The crystalline free base isopropyl alcohol solvate exhibited characteristic reflections corresponding to d-spacings of 20.1, 7.7, 4.7, 11.7, 6.8, 3.9, 5.9, 6.5, and 10.1 d-spacings of angstroms.

One embodiment of the present invention provides a process for preparing a compound of formula I, or a salt, hydrate, solvate, or polymorph thereof,

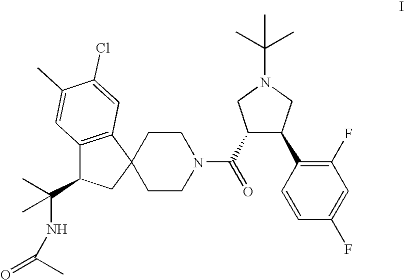

comprising the step of coupling a compound of formula II, or a salt thereof,

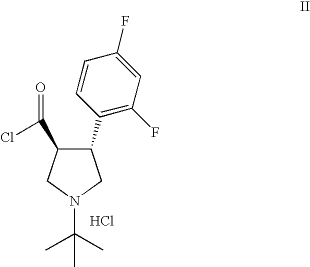

with a compound of formula III,

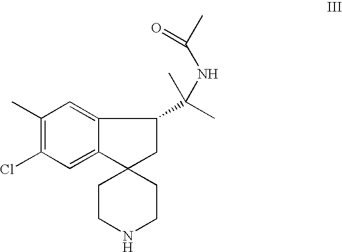

in the presence of an base and a solvent.

In a class of this embodiment, the salt of the compound of formula II is the hydrochloride salt.

In another class of this embodiment, the base is selected from the group consisting of: tertiary amines, such as triethylamine, Hünig's base (diisopropylethylamine), tributylamine, tricyclohexylamine, tetramethylethylenediamine (TMEDA), N-methylpyrrolidine, N-methylmorpholine, (−)-sparteine, diazabicycloundecene (DBU), diazabicyclooctane (DABCO), tetramethylguanidine (TMG), or inorganic bases, such as potassium carbonate, potassium phosphate, sodium carbonate, sodium phosphate. In a subclass of this class, the base is triethylamine.

In another class of this embodiment, the reaction media can be composed of: (1) a single or a mixture of aprotic organic solvents, such as tetrahydrofuran, dimethylformamide, acetonitrile, isopropyl acetate, ethyl acetate, tert-butyl methyl ether, N-methylpyrrolidone, dimethylacetamide, 2-methyltetrahydrofuran, toluene, when organic base is used, or (2) a mixture of aprotic organic solvent above and water, when inorganic base is used. In a subclass of this class, the solvent is tetrahydrofuran. In another subclass of this class, the solvent is tetrahydrofuran and DMF. In another subclass of this class, the base is triethylamine and the solvent is about 20 mole % dimethylformamide in tetrahydrofuran.

In another class of this embodiment, the process further comprises converting compound IV to compound II by converting the acid of compound IV into an acid chloride. In a subclass of this class, the acid of compound IV is converted to the acid chloride of compound II by treatment with oxalyl chloride. In another subclass of this class, compound II is formed or isolated as a salt. In a subclass of this subclass, compound II is formed or isolated as an HCl salt.

In another class of this embodiment, the process further comprises converting compound V to compound III by removing the Boc protecting group of compound V. In a subclass of this class, the Boc protecting group is removed by treatment with an acid in a solvent. In a subclass of this subclass, the acid is HCl. In another subclass of this subclass, the solvent is selected from tetrahydrofuran, isopropyl acetate and isopropyl alcohol. In another subclass of this subclass, the solvent is tetrahydrofuran. In another subclass of this subclass, the acid is concentrated HCl and the solvent is tetrahydrofuran.

In a class of this embodiment, the process further comprises isolating compound I. In another class of this embodiment, the process further comprises isolating compound I as the isopropyl alcohol solvate. In a subclass of this class, compound I, or a solvate thereof, is treated with HCl to form the HCl salt of compound I. In another subclass of this class, the isopropyl alcohol solvate of compound I is treated with HCl to form the HCl salt. In a subclass of this subclass, the HCl salt is a mono HCl salt. In another subclass of this subclass, the mono HCl salt of compound I is crystalline. In another subclass of this subclass, the HCl salt of compound I is dried.

In another class of this embodiment, crystalline anhydrous mono HCl salt of compound I is prepared by a process comprising the steps of:
(a) dissolving the isopropyl alcohol solvate of the compound of formula I in anhydrous acetonitrile to form a suspension;
(b) heating the suspension of step (a) to about 30° C. to form a solution;
(c) filtering the solution of step (b) to form a solution;
(d) adding a solution of HCl in isopropanol to the solution of step (c) at about 20° C. to give a solution;
(e) aging the solution of step (d);
(f) adding isopropyl acetate to the solution of step (d) to form a suspension;
(g) aging the suspension of step (f) at room temperature;
(h) filtering and drying the suspension of step (f); and
(i) drying the suspension of step (f).

In another class of this embodiment, the process further comprises isolating compound I as the mono HCl salt acetonitrile solvate. In a subclass of this class, compound I, or a solvate thereof, is treated with HCl to form the HCl salt of compound I. In another subclass of this class, the isopropyl alcohol solvate of compound I is treated with HCl to form the HCl salt of compound I. In a subclass of this subclass, the HCl salt is a mono HCl salt of compound I. In another subclass of this class, the HCl salt of compound I is crystallized using acetonitrile/isopropyl acetate. In a subclass of this subclass, the mono HCl salt of compound I is crystalline. In another subclass of this subclass, the HCl salt of compound I is dried under a constant stream of N₂ at room temperature overnight to give the mono HCl salt acetonitrile solvate of Compound I.

In another class of this embodiment, the process further comprises isolating compound I as the anhydrous mono HCl salt. In a subclass of this class, compound I, or a solvate thereof, is treated with HCl to form the HCl salt of compound I. In another subclass of this class, the isopropyl alcohol solvate of compound I is treated with HCl to form the HCl salt of compound I. In a subclass of this subclass, the HCl salt is a mono HCl salt of compound I. In another subclass of this class, the HCl salt of compound I is crystallized using acetonitrile/isopropyl acetate. In a subclass of this subclass, the mono HCl salt of compound I is crystalline. In another subclass of this subclass, the HCl salt of compound I is dried in a vacuum oven at 100° C. under constant stream of N₂ at 90-95 torr for 4-5 days to give the anhydrous mono HCl salt of Compound I.

In another class of this embodiment, the process further comprises isolating compound I as the HCl salt hydrate. In a subclass of this class, compound I, or a solvate thereof, is treated with HCl to form the HCl salt of compound I. In another subclass of this class, the HCl salt of compound I is treated with water to form the HCl salt hydrate of compound I. In a subclass of this subclass, the HCl salt is a mono HCl salt. In another subclass of this subclass, the mono HCl salt hydrate of compound I is crystalline. In another subclass of this class, the anhydrous HCl salt of compound I is treated with aqueous KCl to form the HCl salt hydrate of compound I. In a subclass of this subclass, the HCl salt is a mono HCl salt. In another subclass of this subclass, the mono HCl salt hydrate of compound I is crystalline.

Another embodiment of the present invention provides for a method of preventing or treating obesity or an obesity related disorder comprising administering a therapeutically effective amount of a polymorph, free base, hydrate, solvate or salt of Compound I to a subject in need thereof.

The use of a therapeutically effective amount of a polymorph, free base, hydrate, solvate, or salt of Compound I for the manufacture of a medicament useful for the treatment, control, or prevention of obesity, sexual dysfunction, or an obesity-related disorder in a subject in need of such treatment.

The term “N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide” comprises not only the solid form of N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide, but also any amorphous or partially crystalline solid form of N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide, such as glasses, lyophilates, and mixtures thereof, which may be converted to N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide through warming.

Polymorphs are compounds having the same chemical composition but different crystal structures. Polymorphism is the ability of the same chemical substance to exist as different crystalline structures. The compound of structural formula I has been found it exist in at least four polymorphic or crystalline forms, the free base isopropyl alcohol solvate, the anhydrous mono HCl salt, the mono HCl salt hydrate, and the mono HCl salt acetonitrile solvate, each of which can be formed by careful control of the crystallization conditions.

The term “hydrate” is meant to include all full, multiple and partial hydrates of compound I, including, but not limited to, the mono hydrate, hemi-hydrate and bis hydrate.

The term “solvate” is meant to include compound forms containing solvent molecules within the crystal structure of Compound I, or solvent molecules bound to or associated with Compound I, including but not limited to acetonitrile and methanol.

The term “amorphous” refers to solid forms that have no long-range molecular order.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic acid, hydrochloric acid, isethionic acid, lactic acid, maleic acid, malic acid, mandelic acid, methanesulfonic acid, malonic acid, mucic acid, nitric acid, pamoic acid, pantothenic acid, phosphoric acid, propionic acid, succinic acid, sulfuric acid, tartaric acid, p-toluenesulfonic acid, trifluoroacetic acid, and the like. In one embodiment of the present invention, the salts are prepared from the following acids: hydrochloric acid, phosphoric acid, sulfuric acid, L-tartaric acid, succinic acid, hydrobromic acid, L-malic acid, and lactic acid. In another embodiment of the present invention, the salts are prepared from hydrochloric acid.

It will be understood that, as used herein, references to compound I and the compound of Formula I are meant to also include the pharmaceutically acceptable salts, such as the hydrochloride salts, as well as the free base, solvates, hydrates and polymorphs thereof.

In one embodiment of the present invention there is provided a pharmaceutical composition comprising N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I) as a free base, salt, hydrate, solvate or polymorph thereof. In a class of this embodiment, the composition comprises N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I) as the polymorphic free base. In a subclass of this class, the free base is in substantially pure form. In another subclass of this class, the free base is crystalline.

In another class of this embodiment, the composition comprises N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I) as the free base isopropyl alcohol solvate. In a subclass of this class, the free base isopropyl alcohol solvate is in substantially pure form. In another subclass of this class, the free base isopropyl alcohol solvate is crystalline.

In another class of this embodiment, the composition comprises N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I) as the polymorphic anhydrous HCl salt. In a subclass of this class, the anhydrous HCl salt is an anhydrous mono HCl salt. In another subclass of this class, the anhydrous HCl salt is in substantially pure form. In another subclass of this class, the anhydrous mono HCl salt is crystalline.

In another class of this embodiment, the composition comprises N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I) as the polymorphic HCl salt hydrate. In a subclass of this class, the HCl salt hydrate is in substantially pure form. In another subclass of this class, the HCl salt hydrate is crystalline.

In another class of this embodiment, the composition comprises N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I) as the polymorphic HCl salt acetonitrile solvate. In a subclass of this class, the HCl salt acetonitrile solvate is in substantially pure form. In another subclass of this class, the HCl salt acetonitrile solvate is crystalline.

The compounds in the processes of the present invention include stereoisomers, such as optical isomers, diastereomers and geometrical isomers, or tautomers depending on the mode of substitution. The present invention is meant to comprehend all such isomeric forms of the compound in the compositions of the present invention, and their mixtures. All hydrates, solvates, free bases, salts and polymorphic crystalline forms of the above-described compound and their use, including their use in the processes of the instant invention, are encompassed within scope of the instant invention.

Another aspect of the present invention provides pharmaceutical compositions which comprises a polymorph, free base, hydrate, solvate or salt of Compound I and a pharmaceutically acceptable carrier. The pharmaceutical compositions of the present invention comprise compound I as an active ingredient or a pharmaceutically acceptable salt thereof, and may also contain a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the polymorphs, free bases, hydrates, solvates, and salts of Compound I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The polymorphs, free bases, hydrates, solvates, and salts of Compound I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The present invention provides a method for the treatment and/or prevention of obesity and obesity-related disorders in a subject in need thereof comprising administering a therapeutically effective amount of a free base, solvate, hydrate, salt or polymorph of Compound I to the subject in need thereof. The present invention also provides for the use of the free base, hydrates, solvates, salts and polymorphs of Compound I for the manufacture of a medicament for the prevention and/or treatment of obesity and obesity related disorders.

The obesity-related disorders herein are associated with, caused by, or result from obesity. Examples of obesity-related disorders include overeating, binge eating, and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compositions of the present invention are also useful for reducing the risk of secondary outcomes of obesity, such as reducing the risk of left ventricular hypertrophy.

Treatment of obesity and obesity-related disorders refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in subjects in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis.

“Male sexual dysfunction” includes impotence, loss of libido, and erectile dysfunction.

“Erectile dysfunction” is a disorder involving the failure of a male mammal to achieve erection, ejaculation, or both. Symptoms of erectile dysfunction include an inability to achieve or maintain an erection, ejaculatory failure, premature ejaculation, or inability to achieve an orgasm. An increase in erectile dysfunction and sexual dysfunction can have numerous underlying causes, including but not limited to (1) aging, (b) an underlying physical dysfunction, such as trauma, surgery, and peripheral vascular disease, and (3) side-effects resulting from drug treatment, depression, and other CNS disorders.

Treatment of male sexual dysfunction refers to the administration of a compound or combination of the present invention to treat impotence and/or loss of libido, and/or erectile dysfunction in a male mammal in need thereof. One outcome of treatment may be a decrease in impotence. Another outcome of treatment may be an increase in libido. Yet another outcome of treatment may be a decrease in the magnitude or frequency of erectile dysfunction.

Treatment of male sexual dysfunction refers to the administration of a compound or combination of the present invention to treat one or more of the symptoms of male sexual dysfunction in a male mammal in need thereof. One outcome of treatment may be increasing the ability to achieve an erection. Another outcome of treatment may be increasing the ability to maintain an erection. Another outcome of treatment may be reducing ejaculatory failure. Another outcome of treatment may be decreasing premature ejaculation. Yet another outcome of treatment may be increasing the ability to achieve an orgasm.

Prevention of male sexual dysfunction and male erectile dysfunction refers to the administration of the compounds or combinations of the present invention to prevent the symptoms of sexual dysfunction and erectile dysfunction in a male mammal at risk thereof.

“Female sexual dysfunction” can be seen as resulting from multiple components including dysfunction in desire, sexual arousal, sexual receptivity, and orgasm related to disturbances in the clitoris, vagina, periurethral glans, and other trigger points of sexual function. In particular, anatomic and functional modification of such trigger points may diminish the orgasmic potential in breast cancer and gynecologic cancer patients. Treatment of female sexual dysfunction with an MC-4 receptor agonist can result in improved blood flow, improved lubrication, improved sensation, facilitation of reaching orgasm, reduction in the refractory period between orgasms, and improvements in arousal and desire. In a broader sense, “female sexual dysfunction” also incorporates sexual pain, premature labor, and dysmenorrhea.

The terms “administration of” and or “administering” a compound should be understood to mean providing a compound of the invention or a prodrug of a compound of the invention to a subject in need of treatment.

The administration of the compounds of the present invention in order to practice the present methods of therapy is carried out by administering a therapeutically effective amount of the compound to a subject in need of such treatment or prophylaxis. The need for a prophylactic administration according to the methods of the present invention is determined via the use of well known risk factors.

The term “therapeutically effective amount” as used herein means the amount of the active compound that will elicit the biological or medical response in a tissue, system, subject, mammal, or human that is being sought by the researcher, veterinarian, medical doctor or other clinician, which includes alleviation of the symptoms of the disorder being treated. The novel methods of treatment of this invention are for disorders known to those skilled in the art.

The term “prophylactically effective amount” as used herein means the amount of the active compound that will elicit the biological or medical response in a tissue, system, subject, mammal, or human that is being sought by the researcher, veterinarian, medical doctor or other clinician, to prevent the onset of the disorder in subjects as risk for obesity or the disorder.

The therapeutically or prophylactically effective amount, or dosage, of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgement.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compound I, or a polymorph, free base, solvate, hydrate or salt thereof, is administered orally or topically.

The effective dosage of active ingredient employed may vary depending on the particular compound employed, the mode of administration, the condition being treated and the severity of the condition being treated. Such dosage may be ascertained readily by a person skilled in the art.

When treating obesity, in conjunction with diabetes and/or hyperglycemia, or alone, generally satisfactory results are obtained when compound I, or a polymorph, free base, solvate, hydrate or salt thereof, is administered at a daily dosage of from about 0.001 milligram to about 100 milligrams per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating diabetes mellitus and/or hyperglycemia, as well as other diseases or disorders for which compound I is useful, generally satisfactory results are obtained when compound I, or a polymorph, free base, solvate, hydrate or salt thereof, is administered at a daily dosage of from about 0.001 milligram to about 100 milligram per kilogram of animal body weight, preferably given in a single dose or in divided doses two to six times a day, or in sustained release form. In the case of a 70 kg adult human, the total daily dose will generally be from about 0.07 milligrams to about 350 milligrams. This dosage regimen may be adjusted to provide the optimal therapeutic response.

For the treatment of sexual dysfunction, including male erectile dysfunction, compound I, or a polymorph, free base, solvate, hydrate or salt thereof, is given in a dose range of 0.001 milligram to about 100 milligram per kilogram of body weight, preferably as a single dose orally or as a nasal spray.

In the case where an oral composition is employed, a suitable dosage range is, e.g. from about 0.01 mg to about 1500 mg of compound I, or a polymorph, free base, solvate, hydrate or salt thereof, per day, preferably from about 0.1 mg to about 10 mg per day. For oral administration, the compositions are preferably provided in the form of tablets containing from 0.01 to 1,000 mg, preferably 0.01, 0.05, 0.1, 0.5, 1, 2.5, 5, 10, 15, 20, 25, 30, 40, 50, 100, 250, 500, 750, 1000, 1250 or 1500 milligrams of the active ingredient for the symptomatic adjustment of the dosage to the patient to be treated.

For use where a composition for intranasal administration is employed, intranasal formulations for intranasal administration comprising 0.001-10% by weight solutions or suspensions of compound I, or a polymorph, free base, solvate, hydrate or salt thereof, in an acceptable intranasal formulation may be used.

For use where a composition for intravenous administration is employed, a suitable dosage range is from about 0.001 mg to about 100 mg (preferably from 0.01 mg to about 50 mg, more preferably 0.1 mg to 10 mg) of compound I, or a polymorph, free base, solvate, hydrate or salt thereof, per kg of body weight per day. This dosage regimen may be adjusted to provide the optimal therapeutic response. It may be necessary to use dosages outside these limits in some cases.

For the treatment of diseases of the eye, ophthalmic preparations for ocular administration comprising 0.001-1% by weight solutions or suspensions of compound I, or a polymorph, free base, solvate, hydrate or salt thereof, in an acceptable ophthalmic formulation may be used.

The magnitude of prophylactic or therapeutic dosage of compound I, or a polymorph, free base, solvate, hydrate or salt thereof, will, of course, vary depending on the particular compound employed, the mode of administration, the condition being treated and the severity of the condition being treated. It will also vary according to the age, weight and response of the individual patient. Such dosage may be ascertained readily by a person skilled in the art.

X-ray powder diffraction studies are widely used to characterize molecular structures, crystallinity, and polymorphism. The X-ray powder diffraction patterns of the crystalline anhydrous HCl salt of the present invention were generated on a Philips Analytical X'Pert PRO X-ray Diffraction System with PW3040/60 console. A PW3373/00 ceramic Cu LEF X-ray tube K-Alpha radiation was used as the source.

The crystalline anhydrous HCl salt of compound I was further characterized by solid-state carbon-13 nuclear magnetic resonance (NMR) spectra. The solid-state carbon-13 NMR spectra were obtained on a Bruker DSX 500WB NMR system using a Bruker 4 mm H/X/Y CPMAS probe. The carbon-13 NMR spectra utilized proton/carbon-13 cross-polarization magic-angle spinning with variable-amplitude cross polarization, total sideband suppression, and SPINAL decoupling at 100 kHz. The samples were spun at 10.0 kHz, and a total of 1500 scans were collected with a recycle delay of 5 seconds. A line broadening of 10 Hz was applied to the spectra before FT was performed. Chemical shifts are reported on the TMS scale using the carbonyl carbon of glycine (176.03 p.p.m.) as a secondary reference.

Differential scanning calorimetry (DSC) data were acquired using TA Instruments DSC 2910 or equivalent instrumentation was used. Between 1 and 6 mg sample was weighed into an open pan. This pan was then crimped and placed at the sample position in the calorimeter cell. An empty pan was placed at the reference position. The calorimeter cell was closed and a flow of nitrogen was passed through the cell. The heating program was set to heat the sample at a heating rate of 10° C./min to a temperature of approximately 300° C. The heating program was started. When the run was completed, the data were analyzed using the DSC analysis program contained in the system software. The melting endotherm was integrated between baseline temperature points that are above and below the temperature range over which the endotherm was observed. The data reported are the onset temperature, peak temperature and enthalpy.

Thermogravimetric analysis (TGA) data were acquired using a Perkin Elmer model TGA 7 or equivalent instrumentation. Experiments were performed under a flow of nitrogen and using a heating rate of 10° C./min to a maximum temperature of approximately 350° C. After automatically taring the balance, 5 to 20 mg of sample was added to the platinum pan, the furnace was raised, and the heating program started. Weight/temperature data were collected automatically by the instrument. Analysis of the results was carried out by selecting the Delta Y function within the instrument software and choosing the temperatures between which the weight loss was to be calculated. Weight losses are reported up to the onset of decomposition/evaporation.

In the schemes and examples below, various reagent symbols and abbreviations have the following meanings: DMF is dimethylformamide; h is hour(s); HCl is hydrochloric acid; iPA is isopropyl alcohol; iPAc is isopropyl acetate; kg is kilogram; L is liter, mL is milliliter; mol is moles; MTBE is methyl tert-butyl ether; MeCN is acetonitrile; MeOH is methanol; N is normal; min is minute(s); rt or RT is room temperature and THF is tetrahydrofuran.

A representative experimental procedure utilizing the novel process is detailed below. The following Example is provided to illustrate the invention and is not to be construed as limiting the scope of the invention in any manner.

### EXAMPLE 1

### Preparation of N-[1-((3R)-1′-{[(3 S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I) as the Free Base Isopropyl Alcohol Solvate

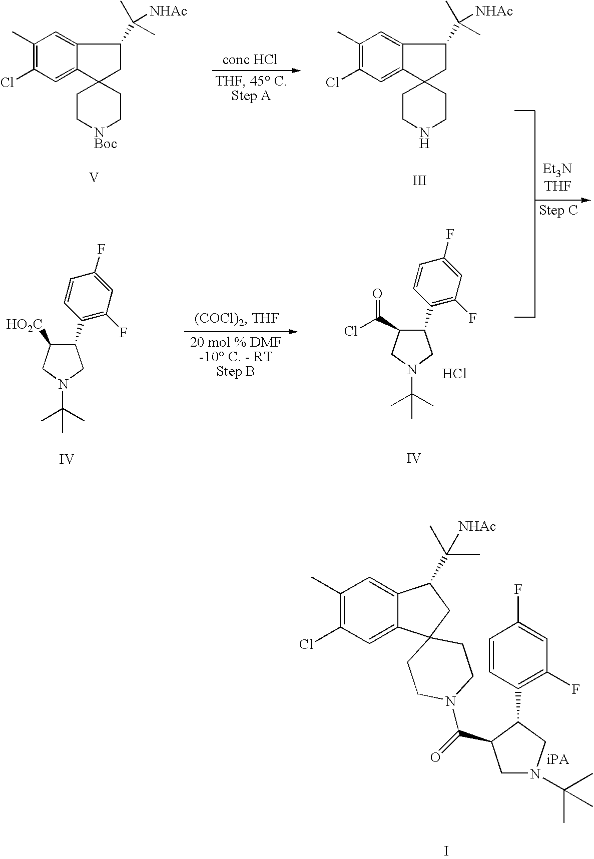

### Step A Preparation of Spiropiperidine III:

To a crude solution of compound V (3.41 kg, 7.839 mol) in THF (20 L) at 40-45° C. was added concentrated HCl (12N, 2.6 L, 31.356 mol) over 1.5 hours with vigorous stirring. The resulting biphasic mixture was aged for 2.5 hours, at which time a complete transformation was observed. After cooling to room temperature, heptane (20 L) and water (17 L) were added to give clear a biphasic solution. The aqueous layer was separated, diluted with 1.5:1 THF:MTBE (17 L) and treated slowly with solid K₂CO₃ (4 kg), followed by NaCl (2 kg). After allowing the layers to settle, the organic layer was separated and azeotropically dried with THF to bring the final volume of 13 L with KF<600 ppm.

### Step B: Preparation of Acid Chloride II:

To a suspension of compound IV (2.55 kg, 9.015 mol) and DMF (122 mL, 1.568 mol) in THF (15 L) at −10° C. was added neat oxalyl chloride (838 mL, 9.407 mol) over 45 minutes. The pale yellowish solution was then warmed to room temperature and aged for 3 hours at which >98.5% consumption of the acid was typically observed. The suspension was used as is in step C.

### Step C: Preparation of the Free Base Isopropyl Alcohol Solvate of Compound I

The suspension of compound II of Step B was cooled back to −20° C. and treated with neat Et₃N (3.83 L, 27.437 mol) over 20 minutes. To the resulting suspension was added over 30 minutes the solution of free spiropiperidine III from Step A in THF. The resulting thick suspension was then allowed to warm to 15-20° C. and aged for 1 hour, at which time a complete transformation was observed. Water (3.8 L) was added to give a homogenous solution, which was then solvent switched to MTBE. The final MTBE solution was sequentially washed once with water (12 L), twice with 10% aqueous K₂CO₃ (2×12 L), and brine (12 L). The solution was then concentrated to remove the bulk of the MTBE and solvent switched to iPA to give a final volume of 20 L. The resulting suspension was heated to reflux to give a clear solution and allowed to cool slowly over 1 hour to 50° C., at which time crystallization started to occur. After further cooling to 40° C. over 30 minutes and aging for an additional 30 minutes, heptane was added over 1 hour at this temperature. The resulting suspension was then cooled to 0° C. and aged overnight. The suspension was then filtered and the wet cake was washed with cold 1:1 iPA:heptane (11 L). The solid was then dried under a stream flow of N₂ atmosphere at room temperature to give the free base isopropyl alcohol solvate of Compound I.

FIG. 7 shows the X-ray diffraction pattern for the crystalline free base isopropyl alcohol solvate of Compound I. The crystalline free base isopropyl alcohol solvate exhibited characteristic reflections corresponding to d-spacings of 20.1, 7.7, 4.7 and angstroms. The crystalline free base isopropyl alcohol solvate was further characterized by reflections corresponding to d-spacings of 11.7, 6.8, and 3.9 angstroms. The crystalline HCl salt acetonitrile solvate was even further characterized by reflections corresponding to 5.9, 6.5, and 10.1 d-spacings of angstroms.

TABLE 1
Powder X-ray diffraction: crystalline free
base isopropyl alcohol solvate of Compound I

| d-spacing (A) | 2θ(2 theta)(degrees) |
|---|---|
| 20.1 | 4.4 |
| 11.7 | 7.6 |
| 10.1 | 8.7 |
| 7.7 | 11.5 |
| 6.8 | 13.0 |
| 6.5 | 13.7 |
| 5.9 | 15.0 |
| 4.7 | 18.8 |
| 3.9 | 22.9 |

Although the crystalline free base isopropyl alcohol solvate of Compound I is characterized by the complete group of angle 2 theta values and d-spacing values listed in Table 1, all the values are not required for such identification. The crystalline free base isopropyl alcohol solvate of Compound I can be identified by the angle theta value in the range of 4.0-5.0°. The crystalline free base isopropyl alcohol solvate of Compound I can be identified by any one of the following angle theta values, or any one of the following groups of angle theta values:

a) 4.4°;
b) 4.4 and 11.5°;
c) 4.4°, 11.5° and 18.8°;
d) 4.4°, 11.5°, 18.8° and 7.6°;
e) 4.4°, 11.5°, 18.8°, 7.6° and 13.0°;
f) 4.4°, 11.5°, 18.8°, 7.6°, 13.0° and 22.9°;
g) 4.4°, 11.5°, 18.8°, 7.6°, 13.0°, 22.9° and 15.0°;
h) 4.4°, 11.5°, 18.8°, 7.6°, 13.0°, 22.9°, 15.0° and 13.7°;
i) 4.4°, 11.5°, 18.8°, 7.6°, 13.0°, 22.9°, 15.0°, 13.7°, and 8.7°.

The crystalline free base isopropyl alcohol solvate of Compound I can also be identified by one or more reflections at d-spacings of: 20.1, 7.7, 4.7, 11.7, 6.8, 3.9, 5.9, 6.5, and 10.1 Å (angstroms) from an X-ray powder diffraction pattern obtained using Cu radiation.

### EXAMPLE 2

### Preparation of the Mono HCl Salt Acetonitrile Solvate of N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I)

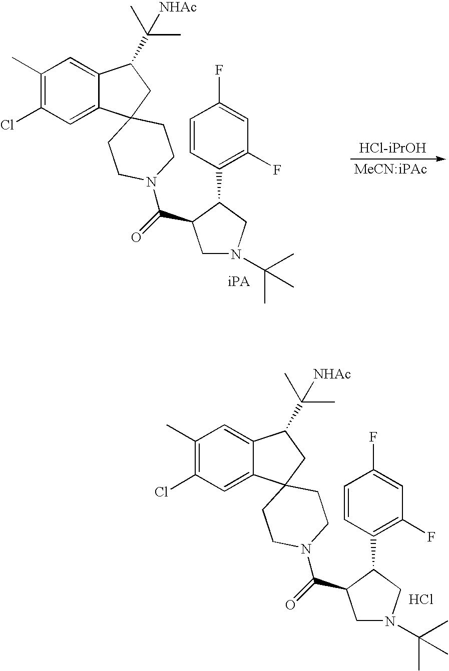

Compound I as the free base isopropyl alcohol solvate (3.60 kg, 6.0 moles, the product of Example 1) was charged with anhydrous acetonitrile (18 L) to give a thin suspension, which upon heating to 30° C. gave a homogenous solution. To the filtered solution was then added a solution of HCl in isopropanol (1.12 L, 6.3 moles, 5.63 N) over 15-25 minutes at 20° C. The resulting solution was then aged for 45 minutes, at which time crystals started to form. After additional aging for 1 hour, isopropyl acetate (11 L) was then added over 1 hour. The resulting suspension was aged for 3 hours at room temperature. The solid was then filtered and the wet cake was washed with cold 1:1 acetonitrile:isopropyl acetate (7.5 L) and dried under a constant stream of N₂ at room temperature overnight to give the crystalline mono HCl salt acetonitrile solvate of Compound I.

FIG. 6 shows the X-ray diffraction pattern for the crystalline mono HCl salt acetonitrile solvate. The crystalline mono HCl salt acetonitrile solvate exhibited characteristic reflections corresponding to d-spacings of 13.58, 6.92, and 5.46 angstroms. The crystalline mono HCl salt acetonitrile solvate was further characterized by reflections corresponding to d-spacings of 8.95, 7.99, and 4.48 angstroms. The crystalline mono HCl salt acetonitrile solvate was even further characterized by reflections corresponding to 3.77, 3.46, and 3.02 d-spacings of angstroms.

TABLE 2
Powder X-ray diffraction: crystalline
mono HCl salt acetonitrile solvate of Compound I

| d-spacing (A) | 2θ(2 theta)(degrees) |
|---|---|
| 13.6 | 6.5 |
| 8.9 | 9.9 |
| 8.0 | 11.1 |
| 6.9 | 12.8 |
| 5.5 | 16.2 |
| 4.5 | 19.8 |
| 3.8 | 23.6 |
| 3.5 | 25.7 |
| 3.0 | 29.6 |

Although the crystalline mono HCl salt acetonitrile solvate of Compound I is characterized by the complete group of angle 2 theta values and d-spacing values listed in Table 2, all the values are not required for such identification. The crystalline mono HCl salt acetonitrile solvate of Compound I can be identified by the angle theta value in the range of 6.0-7.0°. The crystalline mono HCl salt acetonitrile solvate of Compound I can be identified by any one of the following angle theta values, or any one of the following groups of angle theta values:

a) 6.5°;
b) 6.5° and 19.8°;
c) 6.5°, 19.8° and 12.8°;
d) 6.5°, 19.8°, 12.8° and 25.7°;
e) 6.5°, 19.8°, 12.8°, 25.7° and 23.6°;
f) 6.5°, 19.8°, 12.8°, 25.7°, 23.6° and 16.2°;
g) 6.5°, 19.8°, 12.8°, 25.7°, 23.6°, 16.2° and 11.1°;
h) 6.5°, 19.8°, 12.8°, 25.7°, 23.6°, 16.2°, 11.1° and 9.9°;
i) 6.5°, 19.8°, 12.8°, 25.7°, 23.6°, 16.2°, 11.1°, 9.9° and 29.6°.

The crystalline mono HCl salt acetonitrile solvate of Compound I can also be identified by one or more reflections at d-spacings of: 13.6, 4.5, 6.9, 3.5, 3.8, 5.5, 8.0, 8.9, and 3.0 Å (angstroms) from an X-ray powder diffraction pattern obtained using Cu radiation.

### EXAMPLE 3

### Preparation of the Crystalline Anhydrous Mono HCl Salt of N-[1-((3R)-1′-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I)

The crystalline mono HCl salt acetonitrile solvate of Compound I (the product of Example 2 as a semi-dry solid) was further dried in a vacuum oven at 100° C. under constant stream of N₂ at 90-95 torr for 4-5 days to give the crystalline anhydrous mono HCl salt of compound I. The proton magnetic resonance spectrum of Compound I as the anhydrous mono HCl salt was obtained using a Bruker DRX-500 nuclear magnetic resonance (NMR) spectrometer operating at a frequency of 500.13 MHz. The sample concentration was approximately 4.6% (w/v) in DMSO-d₆. The reference compound was DMSO-d₅ (2.50 ppm). Signal assignments are tabulated following the numbered structural formula of Compound I below:

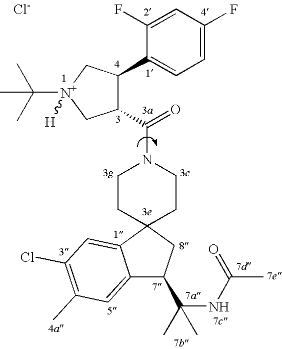

| δ_{H} (ppm) | Multiplicity | Assignment |
|---|---|---|
| 12.14 | br s | N₁H |
| 11.37 | br s | (N₁H) |
| 7.92 | m | C_{6′}H |
| 7.85-7.82 | m | N_{7″c}H, (N_{7″c}H) |
| 7.76 | m | (C_{6′}H) |
| 7.31-7.19 | om | C_{3′}H, C_{5″}H (C_{3′}H, C_{5″}H) |
| 7.17-7.11 | om | C_{5′}H, (C_{5′}H) |
| 6.80 | s | C_{2″}H |
| 6.72 | s | (C_{2″}H) |
| 4.39 | od, J ~ 13 | C_{3g}H |
| 4.29 | d, J = 13.1 | (C_{3g}H) |
| 4.16 | om | C_{7″}H |
| 4.10 | t, J = 9.0 | (C_{7″}H) |
| 4.02-3.90 | om | C₂H, C₃H, (C₃H), C₄H, (C₄H) |
| 3.82-3.56 | om | (C₂H), C_{3c}H, (C_{3c}H), C₅H, (C₅H) |
| 3.33-3.15 | om | C₂H, (C₂H), C₅H, (C₅H), C_{3c}H, (C_{3c}H) |
| 2.74 | m | C_{3g}H |
| 2.65-2.59 | om | (C_{3g}H) |
| 2.28 | s | C_{4a″}H₃, (C_{4a″}H₃) |
| 2.28-2.23 | om | C_{8″}H (C_{8″}H) |
| 1.87 | s | C_{7e″}H₃, (C_{7e″}H₃) |
| 1.56-1.26 | om | C_{8″}H, (C_{8″}H), C_{3f}H₂, (C_{3f}H₂) |

FIG. 1 shows the X-ray diffraction pattern for the crystalline anhydrous mono HCl salt of compound I. The crystalline anhydrous mono HCl salt exhibited characteristic reflections corresponding to d-spacings of 13.58, 6.89, and 5.46 angstroms. The crystalline anhydrous mono HCl salt was further characterized by reflections corresponding to d-spacings of 8.97, 7.96, and 4.46 angstroms. The crystalline anhydrous mono HCl salt was even further characterized by reflections corresponding to d-spacings of 3.86, 3.47, and 3.02 angstroms. FIG. 2 shows the solid-state carbon-13 CPMAS NMR spectrum for the crystalline anhydrous mono HCl salt of compound I. The crystalline anhydrous mono HCl salt exhibited characteristic signals with chemical shift values of 26.8, 43.1, 122.2, and 159.6 p.p.m. Further characteristic of the crystalline anhydrous mono HCl salt are the signals with chemical shift values of 23.8, 43.8, 126.5, and 171.1 p.p.m. The crystalline anhydrous mono HCl salt is even further characterized by signals with chemical shift values of 55.1, 151.7, and 131.6 p.p.m. FIG. 3 shows the differential calorimetry scan for the crystalline anhydrous mono HCl salt of compound I. The crystalline anhydrous mono HCl salt exhibited an endotherm due to melting and decomposition with an onset temperature of 251.6° C., a peak temperature of 256.6° C., and an enthalpy change of 50.0 J/g. It also exhibited another endotherm due to surface water coming out with an extrapolated onset temperature of 32.3° C., a peak temperature of 71.1° C., and an enthalpy change of 38.4 J/g. FIG. 4 shows a characteristic thermogravimetric analysis (TGA) curve for the crystalline anhydrous mono HCl salt of compound I. TGA indicated a weight loss of about 1.4% due to surface water from ambient temperature to about 150° C.

TABLE 3
X-ray powder diffraction: crystalline
anhydrous mono HCl Salt of Compound I

| d-spacing (A) | 2θ(2 theta)(degrees) |
|---|---|
| 13.6 | 6.5 |
| 9.0 | 9.9 |
| 8.0 | 11.1 |
| 6.9 | 12.9 |
| 5.5 | 16.2 |
| 4.5 | 19.9 |
| 3.9 | 23.1 |
| 3.5 | 25.7 |
| 3.0 | 29.6 |

Although the crystalline anhydrous mono HCl Salt of Compound I is characterized by the complete group of angle 2 theta values and d-spacing values listed in Table 3, all the values are not required for such identification. The crystalline anhydrous mono HCl Salt of Compound I can be identified by the angle theta value in the range of 12.0-13.5°. The crystalline anhydrous mono HCl Salt of Compound I can be identified by any one of the following angle theta values, or any one of the following groups of angle theta values:

a) 12.9°;

b) 12.9° and 23.1°;

c) 12.9°, 23.1° and 16.2°;

d) 12.9°, 23.1°, 16.2° and 6.5°;

e) 12.9°, 23.1°, 16.2°, 6.5° and 19.9°;

f) 12.9°, 23.1°, 16.2°, 6.5°, 19.9° and 25.7°;

g) 12.9°, 23.1°, 16.2°, 6.5°, 19.9°, 25.7° and 11.1°;

h) 12.9°, 23.1°, 16.2°, 6.5°, 19.9°, 25.7°, 11.1° and 9.9°;

i) 12.9°, 23.1°, 16.2°, 6.5°, 19.9°, 25.7°, 11.1°, 9.9° and 29.6°.

The crystalline anhydrous mono HCl Salt of Compound I can also be identified by one or more reflections at d-spacings of: 6.9, 3.9, 5.5, 13.6, 4.5, 3.5, 8.0, 9.0 and 3.0_Å (angstroms) from an X-ray powder diffraction pattern obtained using Cu radiation.

### EXAMPLE 4

### Preparation of the Crystalline Mono HCl Salt Hydrate of N-[1-((3R)-1′-{[(3S,4R)-1-tert-butyl-4-(2,4-difluorophenyl)pyrrolidine-3-yl]carbonyl}-6-chloro-5-methyl-2,3-dihydrospiro[indene-1,4′-piperidine]-3-yl)-1-methylethyl]acetamide (Compound I)

The crystalline mono HCl salt hydrate of Compound I can be formed by swishing the anhydrous crystalline mono HCl salt of Compound I in 0.3 M KCl aqueous solution at room temperature for about 12 hours, followed by filtration and washing with water to remove KCl.

Alternatively, the crystalline mono HCl salt hydrate of Compound I can be formed by swishing the amorphous mono HCl salt of Compound I in water at room temperature or 50 C for about 12-16 hours, followed by filtration.

FIG. 5 shows the X-ray diffraction pattern for the crystalline mono HCl salt hydrate of Compound I. The crystalline mono HCl salt hydrate exhibited characteristic reflections corresponding to d-spacings of 9.86, 4.33, and 3.71 angstroms. The crystalline mono HCl salt hydrate was further characterized by reflections corresponding to d-spacings of 4.22, 3.20, and 2.91 angstroms. The crystalline mono HCl salt hydrate was even further characterized by reflections corresponding to d-spacings of 7.16, 5.98, and 5.47 angstroms.

TABLE 4
Powder X-ray diffraction: crystalline
mono HCl salt hydrate of Compound I

| d-spacing (A) | 2θ(2 theta)(degrees) |
|---|---|
| 9.9 | 9.0 |
| 7.2 | 12.4 |
| 6.0 | 14.8 |
| 5.5 | 16.2 |
| 4.3 | 20.5 |
| 4.2 | 21.1 |
| 3.7 | 24.0 |
| 3.2 | 27.9 |
| 2.9 | 30.7 |

Although the crystalline mono HCl salt hydrate of compound I is characterized by the complete group of angle 2 theta values and d-spacing values listed in Table 4, all the values are not required for such identification. The crystalline mono HCl salt hydrate of compound I can be identified by the angle theta value in the range of 20-21. The crystalline mono HCl salt hydrate of Compound I can be identified by any one of the following angle theta values, or any one of the following groups of angle theta values:

a) 20.5°;
b) 20.5° and 21.1°;
c) 20.50°, 21.1° and 16.2°;
d) 20.5°, 21.1°, 16.2° and 24.0°;
e) 20.5°, 21.1°, 16.2°, 24.0° and 9.0°;
f) 20.5°, 21.1°, 16.2°, 24.0°, 9.0° and 30.7°;
g) 20.5°, 21.1°, 16.2°, 24.0°, 9.0°, 30.7° and 27.9°;
h) 20.5°, 21.1°, 16.2°, 24.0°, 9.0°, 30.7°, 27.9°, and 14.8°; and
i) 20.5°, 21.1°, 16.2°, 24.0°, 9.0°, 30.7°, 27.9°, 14.8°, and 12.4°.

The crystalline mono HCl salt hydrate of Compound I can also be identified by one or more reflections at d-spacings of: 4.33, 9.86, 3.71, 4.22, 3.20, 2.91, 7.16, 5.98, and 5.47 Å (angstroms) from an x-ray powder diffraction pattern obtained using Cu radiation.

What is claimed is:

1. A process for preparing a compound of formula I, or a salt, hydrate, solvate or polymorph thereof, 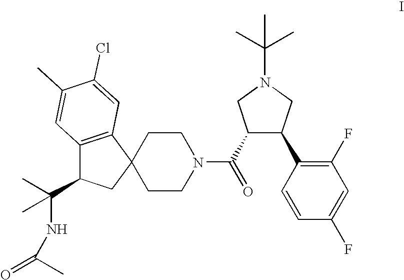
comprising the step of coupling a compound of formula II, or a salt thereof, 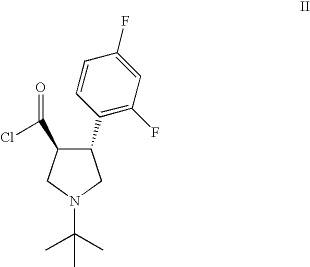
with a compound of formula III, 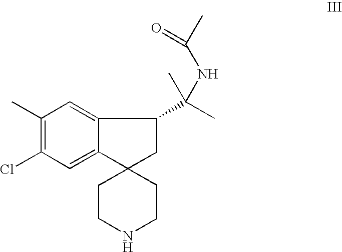
in the presence of a base and a solvent.

2. The process of claim 1 wherein the salt of compound II is an HCl salt.

3. The process of claim 1 wherein the base is triethylamine, and the solvent is a mixture of dimethylformamide and THF.

4. The process of claim 1 wherein the salt of the compound of formula I is the hydrochloric acid salt, or a solvate or hydrate thereof.

5. The process of claim 1 wherein the compound of formula I is the free base, or a solvate thereof.

6. The process of claim 1 wherein the compound of formula I is further isolated as a polymorph selected from the group consisting of:
(1) a free base isopropyl alcohol solvate of a compound of formula I characterized by the X-ray powder diffraction pattern of FIG. 7;
(2) an anhydrous mono HCl salt of a compound of formula I characterized by the X-ray powder diffraction pattern of FIG. 1;
(3) a mono HCl salt hydrate of a compound of formula I characterized by the X-ray powder diffraction pattern of FIG. 5; and
(4) a mono HCl salt acetonitrile solvate of a compound of formula I characterized by the X-ray powder diffraction pattern of FIG. 6.

7. A compound which is the crystalline free base isopropyl alcohol solvate of a compound of formula I: 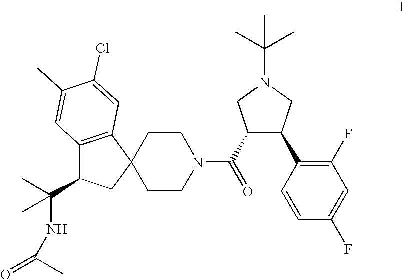
having an X-ray powder diffraction pattern obtained using Cu radiation characterized by a reflection at a d-spacing of 20.1 angstroms, and at least one reflection at a d-spacing selected from the group consisting of: 7.7, 4.7, 11.7, 6.8, 3.9, 5.9, 6.5, and 10.1 angstroms.

8. A compound which is the crystalline mono HCl salt hydrate of a compound of formula I: 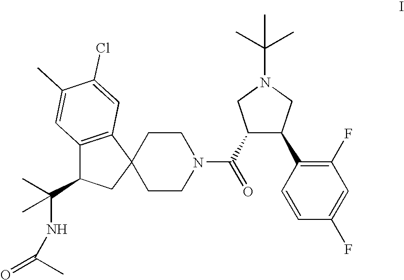
having an X-ray powder diffraction pattern obtained using Cu radiation characterized by a reflection at a d-spacing of 4.33 angstroms, and at least one reflection at a d-spacing selected from the group consisting of: 9.86, 3.71, 4.22, 3.20, 2.91, 7.16, 5.98, and 5.47 angstroms.

9. A compound which is the crystalline mono HCl salt acetonitrile solvate of a compound of formula I: 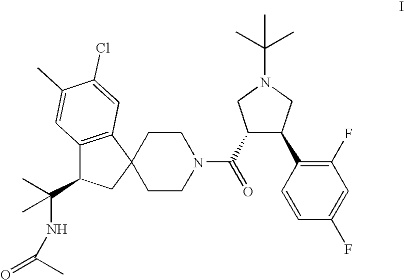
having an X-ray powder diffraction pattern obtained using Cu radiation characterized by a reflection at a d-spacing of 13.6 angstroms, and at least one reflection at a d-spacing selected from the group consisting of: 4.5, 6.9, 3.5, 3.8, 5.5, 8.0, 8.9, and 3.0 d-spacings of angstroms.

10. A compound which is the crystalline anhydrous mono HCl salt of a compound of formula I:

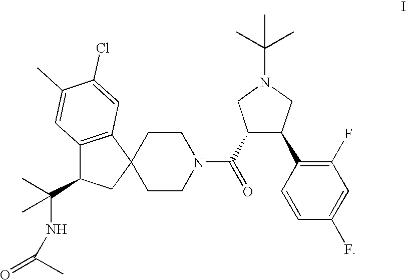

11. A compound in accordance with claim 10 characterized as having an X-ray powder diffraction pattern obtained using Cu radiation containing an angle 2 theta value of 12.5-13.5°.

12. A compound in accordance with claim 10 characterized as having an X-ray powder diffraction pattern obtained using Cu radiation containing the following angle 2 theta values: 12.9° and at least one angle theta value selected from the group consisting of: 23.1°, 16.2°, 6.5°, 19.9°, 25.7°, 11.1°, 9.9° and 29.6°.

13. A compound in accordance with claim 10 characterized as having an x-ray powder diffraction pattern obtained using Cu radiation characterized by a reflection at a d-spacing of 6.9 angstroms, and at least one reflection at a d-spacing selected from the group consisting of: 3.9, 5.5, 13.6, 4.5, 3.5, 8.0, 9.0 and 3.0 angstroms.

14. A compound in accordance with claim 10 characterized as having a differential scanning calorimetry (DSC) peak melting temperature of about 256.6° C.

15. An anhydrous mono HCl salt of a compound of formula I in accordance with claim 10 characterized by the X-ray powder diffraction pattern of FIG. 1.

16. A compound in accordance with claim 10 characterized by the carbon-13 cross-polarization magic-angle spinning nuclear magnetic resonance spectrum of FIG. 2.

17. A pharmaceutical composition comprising a therapeutically effective amount of the crystalline anhydrous mono HCl salt of a compound of formula I in accordance with claim 10, in combination with a pharmaceutically acceptable carrier.

18. A method of treating obesity, diabetes, male erectile dysfunction, male sexual dysfunction, female sexual dysfunction, or an obesity-related disorder in a subject in need thereof comprising administering to the subject a therapeutically effective amount of the anhydrous mono HCl salt of a compound of formula I in accordance with claim 10.

19. A crystalline anhydrous mono HCl salt of Compound I in accordance with claim 10 prepared by a process comprising the steps of:
(a) dissolving the isopropyl alcohol solvate of the compound of formula I in anhydrous acetonitrile to form a suspension;
(b) heating the suspension of step (a) to about 30° C. to form a solution;
(c) filtering the solution of step (b) to form a solution;
(d) adding a solution of HCl in isopropanol to the solution of step (c) at about 20° C. to give a solution;
(e) aging the solution of step (d) to form an aged solution;
(f) adding isopropyl acetate to the aged solution of step (e) to form a suspension;
(g) aging the suspension of step (f) at room temperature to form an aged suspension;
(h) filtering the aged suspension of step (g) to give a filtrate; and
(i) drying the filtrate of step (h).
